(19) United States
(12) Patent Application Publication
(10) Pub. No.: US 2023/0127498 A1
(43) Pub. Date: Apr. 27, 2023

(54) TREATMENT OF RESPIRATORY DISORDERS

(71) Applicant: Terns, Inc., Foster City, CA (US)

(72) Inventors: Martijn FENAUX, San Mateo, CA (US); Christopher T. JONES, Foster City, CA (US); Erin K. QUIRK, Foster City, CA (US)

(21) Appl. No.: 17/914,163

(22) PCT Filed: Mar. 25, 2021
(86) PCT No.: PCT/US2021/024239

§ 371 Date: Sep. 23, 2022

Related U.S. Application Data
(60) Provisional application No. 62/994,617, filed on Mar. 25, 2020.

(51) Int. Cl. A61K 31/496; A61K 9/00; A61P 11/00

(52) U.S. Cl. CPC A61K 31/496; A61K 9/0043; A61P 11/00

(57) ABSTRACT

Provided herein are compounds for use in treating respiratory disorders.

### CROSS-REFERENCE TO RELATED APPLICATION

This application claims priority to and the benefit of U.S. Provisional Application No. 62/994,617, filed on Mar. 25, 2020, the entire contents of which are incorporated herein by reference.

### FIELD

This invention relates to methods and compositions for treating an individual with a respiratory disease.

### BACKGROUND

Acute respiratory distress syndrome (ARDS) is the most severe form of acute lung injury and is characterized by bilateral pulmonary infiltrates and severe hypoxemia. Inflammation is a key driver of disease mediated in part by the secretion of cytokines and other inflammatory mediators by local epithelial cells, triggering recruitment of neutrophils, T-cells, and activated macrophages into the inflamed lung tissue and alveolar space. Gonzales J N. Austin J Vasc. Med. 2015 Jun. 4; 2(1). ARDS can be caused by a variety of insults including viral, bacterial, and parasite infections including SARS-CoV-2, SARS-CoV, MERS-CoV, Influenza virus, Metapneumovirus, Varicella zoster virus, Streptococcus pneumoniae, Haemophilus influenzae, Mycobacterium tuberculosis and malaria. Kyung-Yil L. Int. J. Mol. Sci. 2017, 18, 388; doi:10.3390/ijms18020388. ARDS can also be caused by breathing high concentrations of smoke or chemical fumes, aspirating vomit or near-drowning episodes. Many people with ARDS will not survive, and those that do can have long lasting damage to their lungs (e.g., pulmonary fibrosis). Alberts W. M. American College of Chest Physicians. 1983. 84 (3) p. 272-4.

Semicarbazide-sensitive amine oxidase (SSAO), also known as VAP-1/AOC3, is widely expressed in a variety of tissues including the liver, adipose, and lung, where it functions as an ectoenzyme to breakdown primary amines (e.g. methylamine) into hydrogen peroxide (H₂O₂) and aldehydes. In addition, membrane-bound SSAO functions as an adhesion molecule to facilitate binding and transmigration of leukocytes from the vasculature to the sites of inflammation. The expression of SSAO is elevated in a variety of inflammatory diseases, suggesting inhibitors of SSAO may provide a clinical benefit. Significant elevation of SSAO expression has been measured in patients with ARDS. The excessive accumulation of pro-inflammatory cells in the lung tissue and alveolar space, especially neutrophils, is the driver of the immunopathological mechanism of ARDS (Tsui P. T. Emerging Infectious Diseases. 2003. Vol. 9, No. 9, Franks T. J. Human Pathology. 2003. Volume 34, No. 8. Liao M. (ePub ahead of print) https://doi.org/10.1101/2020.02.23.20026690, Tian S. Journal of Thoracic Oncology (ePub ahead of print) https://doi.org/10.1016/j.jtho.2020.02.010.

In models of acute lung disease, treatment with the potent SSAO inhibitor BI-1467335 (formerly, PX-4728A), reduced LPS-induced neutrophil lung migration and cytokine-induced cell migration by diminishing leukocyte rolling and adherence. Schilter, H. C. Respir. Res. 2015, 16, 42. A related SSAO inhibitor. PXS-4681A, also reduced leukocyte trafficking and cytokine production in both LPS- and carrageenan-induced mouse models of lung inflammation. Foot, J. J. Pharmacol. Exp. Ther. 2013 347(2) 365-374. In these studies, the effects of PXS-4681A were comparable to treatment with the anti-inflammatory glucocorticoid, dexamethasone. In models of chronic obstructive pulmonary disease (COPD) and acute cigarette smoke-induced lung disease, treatment with BI-1467335 suppressed airway infiltration of inflammatory immune cells, reduced pulmonary fibrosis, and improved lung function. Jarnicki, A. G. Br. J. Pharmacol. 2016, 173, 3161-3175. Finally, in a model to mimic viral exacerbation of asthmatic response, BI-1467335 treatment reduced inflammatory cellular infiltrates and airway hyperreactivity in mice sensitized with house dust mite (HDM) and subsequently challenged with HDM and rhinovirus infection. Schilter, H. C. Respir. Res. 2015, 16, 42. However, BI-1467335 retains off-target activity against MAO-B, which may present a safety concern in certain patients.

Accordingly, there is a need for an improved treatment of respiratory disorders, such as ARDS, using a SSAO inhibitor, such as a selective SSAO inhibitor over MAO-A/B.

### BRIEF SUMMARY

Provided herein are compositions and methods for the treatment of respiratory disorders, such as ARDS.

### BRIEF DESCRIPTION OF THE FIGURES

FIG. 1A shows the plasma SSAO activity compared to baseline of healthy volunteers administered a single dose of placebo or 1, 3, 6, or 10 mg of the compound of Formula 5 at 4 hours and 168 hours post dose.

FIG. 1B shows a time course of SSAO activity compared to baseline of healthy volunteers administered a single dose of placebo or 1, 3, 6, or 10 mg of the compound of Formula 5.

FIG. 1C shows a time course of the level of the compound of Formula 5 after a single dose of placebo or 1, 3, 6, or 10 mg in healthy volunteers.

FIG. 1D shows a time course of the level of plasma methylamine after a single dose of placebo or 1, 3, 6, or 10 mg of the compound of Formula 5 in healthy volunteers.

FIG. 2A shows body weight changes of mice dosed with a compound of Formula 5, oseltamivir, or a combination therapy of a compound of Formula 5 and oseltamivir as a prophylactic or a therapeutic relative to healthy and vehicle controls in a lethal influenza challenge model.

FIG. 2B shows lung viral titer of mice dosed with a compound of Formula 5, oseltamivir, or a combination therapy of a compound of Formula 5 and oseltamivir as a prophylactic or a therapeutic relative to healthy and vehicle controls on day 6 of the lethal influenza challenge model.

FIG. 2C, FIG. 2D, FIG. 2E, FIG. 2F, FIG. 2G, FIG. 2H, FIG. 2I, FIG. 2J, FIG. 2K, FIG. 2L, and FIG. 2M show cytokine levels in bronchoalveolar lavage fluid (BALF) of mice dosed with a compound of Formula 5, oseltamivir, or a combination therapy of a compound of Formula 5 and oseltamivir as a prophylactic or a therapeutic relative to healthy and vehicle controls on day 14 of the lethal influenza challenge model.

FIG. 2N shows cell infiltrates in BALF of mice dosed with a compound of Formula 5, oseltamivir, or a combination therapy of a compound of Formula 5 and oseltamivir as a prophylactic or a therapeutic relative to healthy and vehicle controls on day 14 of the lethal influenza challenge model.

FIG. 2O shows lung histopathology scoring of mice dosed with a compound of Formula 5, oseltamivir, or a combination therapy of a compound of Formula 5 and oseltamivir as a prophylactic or a therapeutic relative to healthy and vehicle controls on day 14 of the lethal influenza challenge model.

FIG. 2P shows lung fibrosis scoring of mice dosed with oseltamivir or a combination therapy of a compound of Formula 5 and oseltamivir as a prophylactic or a therapeutic relative to healthy and vehicle controls on day 14 of the lethal influenza challenge model.

### DETAILED DESCRIPTION

Provided herein are methods of treating respiratory diseases, such as Acute Respiratory Distress Syndrome (ARDS), comprising administering an anti-inflammatory agent. In some embodiments, the method comprises administering a SSAO inhibitor. In some embodiments, the SSAO inhibitor is a selective SSAO inhibitor. In some embodiments, the method comprises administering a compound as described herein.

### Definitions

As used herein, the following definitions shall apply unless otherwise indicated. Further, if any term or symbol used herein is not defined as set forth below, it shall have its ordinary meaning in the art.

“Comprising” is intended to mean that the compositions and methods include the recited elements, but not excluding others. “Consisting essentially of” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of, e.g., other ingredients and substantial method steps recited. Embodiments defined by each of these transition terms are within the scope of this invention.

“Combination therapy” or “combination treatment” refers to the use of two or more drugs or agents in treatment, e.g., the use of a compound of Formula 1 or a pharmaceutically acceptable salt thereof together with a second agent useful to treat respiratory disorders (such as ARDS), and symptoms and manifestations of each thereof is a combination therapy. Administration in “combination” refers to the administration of two agents (e.g., a compound of Formula 1, and a second agent) in any manner in which the pharmacological effects of both manifest in the individual at the same time. Thus, administration in combination does not require that a single pharmaceutical composition, the same dosage form, or even the same route of administration be used for administration of both agents or that the two agents be administered at precisely the same time. Both agents can also be formulated in a single pharmaceutically acceptable composition. A non-limiting example of such a single composition is an oral composition or an oral dosage form. For example, and without limitation, it is contemplated that a compound of Formula 1, such as a compound of Formula 5, can be administered in combination therapy with a second agent in accordance with the present disclosure.

“Second agent” as used herein refers to an agent, which is other than a compound of Formula 1, or a pharmaceutically acceptable salt thereof, and which is useful in a method described herein. The term “second” in connection with “second agent” is meant as a term to distinguish the agent from a compound of Formula 5 or a pharmaceutically acceptable salt or enantiomer thereof, and is not intended to signify an order of administration.

The term “excipient” as used herein means an inert or inactive substance that may be used in the production of a drug or pharmaceutical, such as a tablet containing a compound of the invention as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch de, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose de, sorbitol, sucrose de, etc.; and wet granulation agents include, e.g., calcium carbonate, maltodextrin, microcrystalline cellulose, etc.

“Individual” refers to mammals and includes humans and non-human mammals. Examples of patients include, but are not limited to mice, rats, hamsters, guinea pigs, pigs, rabbits, cats, dogs, goats, sheep, cows, and humans. In some embodiments, individual refers to a human.

“Pharmaceutically acceptable” refers to safe and non-toxic, preferably for in vivo, more preferably, for human administration.

“Pharmaceutically acceptable salt” refers to a salt that is pharmaceutically acceptable. A compound described herein may be administered as a pharmaceutically acceptable salt.

“Salt” refers to an ionic compound formed between an acid and a base. When the compound provided herein contains an acidic functionality, such salts include, without limitation, alkali metal, alkaline earth metal, and ammonium salts. As used herein, ammonium salts include, salts containing protonated nitrogen bases and alkylated nitrogen bases. Exemplary and non-limiting cations useful in pharmaceutically acceptable salts include Na, K, Rb, Cs, NH₄, Ca, Ba, imidazolium, and ammonium cations based on naturally occurring amino acids. When the compounds utilized herein contain basic functionality, such salts include, without limitation, salts of organic acids, such as carboxylic acids and sulfonic acids, and mineral acids, such as hydrogen halides, sulfuric acid, phosphoric acid, and the like. Exemplary and non-limiting anions useful in pharmaceutically acceptable salts include oxalate, maleate, acetate, propionate, succinate, tartrate, chloride, sulfate, bisulfate, mono-, di-, and tribasic phosphate, mesylate, tosylate, and the like.

“Therapeutically effective amount” or dose of a compound or a composition refers to that amount of the compound or the composition which results in reduction or inhibition of symptoms or a prolongation of survival in a patient. The results may require multiple doses of the compound or the composition.

As used herein, “treatment” or “treating” is an approach for obtaining beneficial or desired results including clinical results. For purposes of this invention, beneficial or desired results include, but are not limited to, one or more of the following: decreasing one or more symptoms resulting from the disease or disorder, diminishing the extent of the disease or disorder, stabilizing the disease or disorder (e.g., preventing or delaying the worsening of the disease or disorder), delaying the occurrence or recurrence of the disease or disorder, delaying or slowing the progression of the disease or disorder, ameliorating the disease or disorder state, providing a remission (whether partial or total) of the disease or disorder, decreasing the dose of one or more other medications required to treat the disease or disorder, enhancing the effect of another medication used to treat the disease or disorder, delaying the progression of the disease or disorder, increasing the quality of life, and/or prolonging survival of a patient. Also encompassed by “treatment” is a reduction of pathological consequence of the disease or disorder. The methods of the invention contemplate any one or more of these aspects of treatment.

As used herein. “about” a parameter or value includes and describes that parameter or value per se. For example, “about X” includes and describes X per se.

The terms “optional” or “optionally” as used throughout the specification means that the subsequently described event or circumstance may but need not occur, and that the description includes instances where the event or circumstance occurs and instances in which it does not. For example, “the nitrogen atom is optionally oxidized to provide for the N-oxide (N→O) moiety” means that the nitrogen atom may but need not be oxidized, and the description includes situations where the nitrogen atom is not oxidized and situations where the nitrogen atom is oxidized.

### Compounds

The present invention provides compounds of Formula 1:

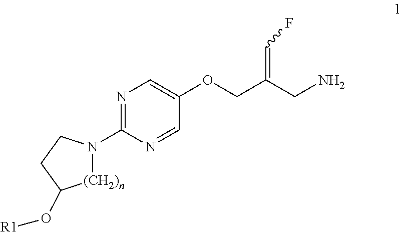

where n is 1 or 2; and R1 is H or —CH₃; or a pharmaceutically acceptable salt thereof.

The bond to fluorine, which is illustrated as [symbol] , indicates that the fluorine atom and the methoxypyrimidine group can be either Z (zusammen, together) or E (entgegen, opposite) relative to each other (Brecher, J., et al., “Graphical Representation of Stereochemical Configuration”. Pure and Appl. Chem, 2006, 78(10) 1897, at 1959). The structure illustrated by Formula 1 includes compounds with the Z stereochemical configuration, the E stereochemical configuration, or a mixture of compounds in the Z or E stereochemical configurations. Preferred compounds of the invention have the E stereochemical configuration.

In one form, the present invention provides compounds of Formula 1 as a free base. In other form, the present invention provides compounds of Formula 1 as acid addition salts, such as a mono or di HCl addition salt(s) or a sulfonate salt, preferable a 4-methylbenzenesulfonate (a tosylate salt).

In one form, the present invention provides a compound of Formula 2:

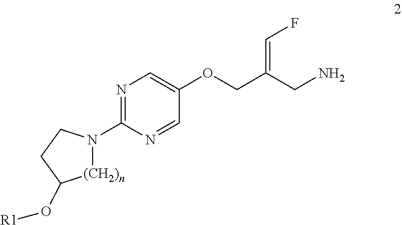

where n is 1 or 2; and R1 is H or —CH₃; or a pharmaceutically acceptable salt thereof.

In another form, the present invention provides a compound of Formula 3:

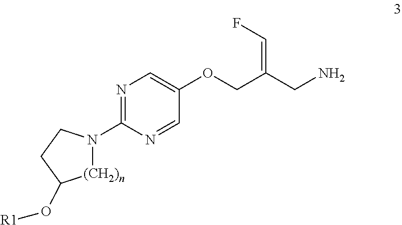

where n is 1 or 2; and R1 is H or —CH₃; or a pharmaceutically acceptable salt thereof.

In one embodiment, the present invention provides a compound according to one of Formulae 1, 2, and 3 where n is 1, or a pharmaceutically acceptable salt thereof. In another embodiment, the present invention provides a compound according to one of Formulae 1, 2, and 3 where n is 2, or a pharmaceutically acceptable salt thereof.

In another embodiment, the present invention provides a compound according to one of Formulae 1, 2, and 3 where R1 is H, or a pharmaceutically acceptable salt thereof. In yet another embodiment, the present invention provides a compound according to one of Formulae 1, 2, and 3 where R1 is —CH₃, or a pharmaceutically acceptable salt thereof.

In another form, the present invention provides a compound of Formula 4

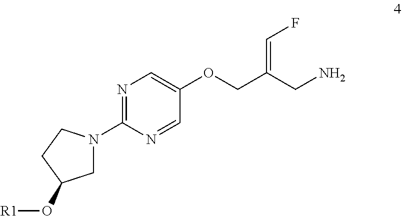

where R1 is H or —CH₃, or a pharmaceutically acceptable salt thereof.

In another form, the present invention provides a compound according to of Formula

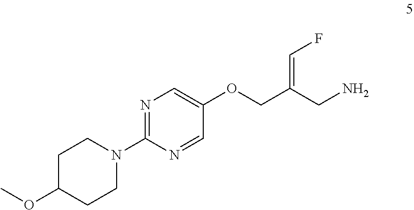 [SMILES: COC1CCN(c2ncc(OC/C(=C/F)CN)cn2)CC1]

or a pharmaceutically acceptable salt thereof. In one embodiment, the compound of Formula 5 is provided as an acid addition salt. Preferably the acid addition salt is a mono or di HCl addition salt or a sulfonate salt, such as a methanesulfonic acid or 4-methylbenzenesulfonic acid addition salt to provide a mesylate salt or a 4-methylbenzenesulfonate (tosylate) salt. In one embodiment, the compound of Formula 5 is provided as a tosylate salt.

In another form, the present invention provides a pharmaceutical composition comprising a compound according to any one of Formulae 1 to 5, or a pharmaceutically acceptable salt thereof, and a pharmaceutically acceptable carrier, diluent, or excipient. In one embodiment, the pharmaceutical composition comprises a compound according to Formula 5, or a pharmaceutically acceptable salt thereof. Preferably the pharmaceutically acceptable anion(s) for the salt is a mono or di chloride, mesylate or a 4-methylbenzenesulfonate (tosylate). In one embodiment, the compound of Formula 5 is provided as a tosylate salt.

### Methods

### Treatment of Respiratory Disorders

Provided herein are methods of treating respiratory diseases, such as Acute Respiratory Distress Syndrome (ARDS), comprising administering an effective amount of an anti-inflammatory agent. In some embodiments, the method comprises administering a SSAO inhibitor. In some embodiments, the compound is an SSAO inhibitor that is selective for SSAO over MAO-A and MAO-B. In some embodiments, the compound is a selective SSAO inhibitor that does not inhibit MAO-A and MAO-B. In some embodiments, the method comprises administering a compound as described herein. In some embodiments, the method comprises administering an effective amount of compound of Formula 5, or a tosylate salt thereof.

In some embodiments, provided herein are methods for treating a respiratory disease or disorder comprising administering to an individual an effective amount of a compound as provided herein. In some embodiments, the method comprises treating an individual with ARDS. In some embodiments, the method comprises treating an individual with pneumonia. In some embodiments, the method comprises treating an individual with chronic obstructive pulmonary disease (COPD). In some embodiments, the method comprises treating an individual with lung cancer. In some embodiments, the method comprises treating an individual with asthma. In some embodiments, the method comprises treating an individual with acute pneumonitis. In some embodiments, the method comprises treating smoke inhalation in an individual. In some embodiments, the method comprises treating an individual with pulmonary fibrosis. In some embodiments, the method comprises treating an individual with reduced lung function.

Acute respiratory distress syndrome (ARDS) occurs when fluid builds up in the tiny, elastic air sacs (alveoli) in the lungs. The fluid keeps the lungs from filling with enough air, which means less oxygen reaches the bloodstream. This deprives the organs of the oxygen they need to function. ARDS can be diagnosed based upon a physical examination. A chest X-ray can reveal which parts of the lungs and how much of the lungs have fluid in them and whether the heart is enlarged. A computerized tomography (CT) scan combines X-ray images taken from many different directions into cross-sectional views of internal organs. CT scans can provide detailed information about the structures within the heart and lungs. A test using blood can measure the oxygen level. Other types of blood tests can check for signs of infection or anemia. Electrocardiogram can also be used to detect ARDS by measuring electrical activity in the heart. Echocardiogram is also used to diagnosis ARDS by revealing problems with the structures and the function of the heart. Symptoms of ARDS include shortness of breath, labored and unusually rapid breathing, low blood pressure, confusion and extreme tiredness.

Accordingly, in some embodiments, provided herein is a method of treating ARDS in an individual comprising administering a compound of the present invention, or a pharmaceutically acceptable salt thereof, to an individual in need thereof. In some embodiments, the method comprises administering an effective amount of compound of Formula 5, or a pharmaceutically acceptable salt thereof.

In some embodiments, the method comprises reducing one or more complications arising from ARDS. In some embodiments, blood clots, collapsed lung (pneumothorax), infection, and/or pulmonary fibrosis are reduced. In some embodiments, cognitive problems, such as memory loss and thinking clearly, are reduced. In some embodiments, tiredness and weakness are reduced.

In some embodiments, the method comprises treating a viral infection comprising administering a compound of the present invention. In some embodiments, the method comprises treating a bacterial infection. In some embodiments, the method comprises treating SARS-CoV-2, SARS-CoV, MERS-CoV, Influenza virus, Metapneumovirus, Varicella zoster virus, Streptococcus pneumoniae, Haemophilus influenzae, Mycobacterium tuberculosis, or malaria.

In some embodiments, the respiratory disordered to be treated is caused by a pathogen. In some embodiments, the respiratory disorder is caused by a virus. In some embodiments, the respiratory disorder is caused by a parasite. In some embodiments, the respiratory disorder is caused by SARS-CoV-2, SARS-CoV, MERS-CoV, Influenza virus. Metapneumovirus, Varicella zoster virus, Streptococcus pneumoniae, Haemophilus influenzae, Mycobacterium tuberculosis, or Malaria. In some embodiments, the respiratory disorder is caused by COVID-19.

In some embodiments the respiratory disorder to be treated is caused by an environmental factor. In some embodiments the respiratory disorder is caused by breathing high concentrations of smoke or chemical fumes, aspirating vomit, or a near-drowning episode.

In some embodiments, the respiratory disorder to be treated is caused by an underlying condition. In some embodiments, the repository disorder is caused by sepsis. In some embodiments, the respiratory disorder is caused by head, chest, or other major injury. In some embodiments, the respiratory disorder is caused by pancreatitis. In some embodiments, the respiratory disorder is caused by a blood transfusion. In some embodiments, the respiratory disorder is caused by a burn.

In some embodiments, provided herein are methods for reducing one or more symptoms of a respiratory disease comprising administering an effective amount of compound as provided herein. In some embodiments, difficulty breathing is reduced. In some embodiments, coughing is reduced. In some embodiments, chest pain is reduced. In some embodiments, mucus level is reduced. In some embodiments, the frequency or severity of coughing up blood is reduced. In some embodiments, the level of frequency of noisy breathing is reduced. In some embodiments, pulmonary fibrosis is reduced. In some embodiments, lung function is improved. In some embodiments, the methods provided herein increase survival time of an individual with a respiratory disease. In some embodiments the method comprises administering an effective amount of a compound of Formula 5, or a pharmaceutically acceptable salt thereof.

Also provided herein is a method of reducing inflammation in the lungs of an individual comprising administering an effective amount of a compound provided herein to the individual. In some embodiments, one or more markers of lung inflammation is reduced. In some embodiments, airway infiltration of inflammatory immune cells is reduced upon treatment. In some embodiments, the level of myeloid cells is decreased. In some embodiments, the level of one or more immune cells or lymphocytes in the lungs of the individual is decreased. In some embodiments, the level of leukocytes including but not limited to neutrophils and macrophages in the lungs is decreased.

In some embodiments, the level of neutrophils in the lungs is reduced by at least 5%, at least 10%, at least 20%, at least 30%, at least 40%, at least 50% or more upon treatment with a compound provided herein. In some embodiments, the level of neutrophils in the lungs is reduced 5% to 50%, 5% to 40%, or 5% to 20%.

In some embodiments, the methods provided herein comprise administering a compound of the present invention to an individual (such as human) via various routes, such as parenterally, intravenously, intra-arterial, intraperitoneal, intrapulmonary, oral, inhalation, intramuscular, intra-tracheal, subcutaneous, intraocular, intrathecal, or transdermal. For example, the compound can be administered by inhalation to treat conditions of the respiratory tract. In some embodiments, the compound is administered intra-nasally.

In some embodiments, provided herein is a method of treating a disease comprising selectively inhibiting SSAO by administering a compound provided herein. Accordingly, in some embodiments, MAO-A (Monoamine oxidase A) is not inhibited. In some embodiments, MAO-B (Monoamine oxidase B) is not inhibited. In some embodiments MAO-A and MAO-B are not inhibited.

In some embodiments, the IC₅₀ for the compound is at least 100-fold lower for SSAO than for MAO-A and/or MAO-B. In some embodiments, the IC₅₀ for the compound is at least 1,000-fold lower for SSAO than for MAO-A and/or MAO-B. In some embodiments, the IC₅₀ for the compound is at least 10,000-fold lower for SSAO than for MAO-A and/or MAO-B. In some embodiments, the IC₅₀ for the compound is between 100 to 10,000-fold lower for SSAO than for MAO-A and/or MAO-B. In some embodiments, the IC₅₀ for the compound is between 100 to 1,000-fold lower for SSAO than for MAO-A or MAO-B. In some embodiments, the IC₅₀ for the compound is at least 100-fold or at least 1,000-fold or at least 10,000-fold or between 100 to 10,000-fold or between 100 to 1,000-fold lower for SSAO than for MAO-A and for MAO-B.

Also provided are methods of inhibiting SSAO activity in an individual for a period of time comprising administering to the individual a once daily dose of a compound described herein (such as the compound of Formula 5) for a first period of time, followed by a second period of time in which administration of the compound is discontinued, wherein the SSAO inhibitory activity is maintained during both the first and the second period of time. In some embodiments, the first and second periods of time are each one-week periods. For example, provided herein is a method of inhibiting SSAO activity in an individual for a period of 14 days comprising administering to the individual a once daily dose of a compound described herein (such as the compound of Formula 5) for a first 7 days, followed by discontinued administration of the compound for the following 7 days, wherein the SSAO inhibitory activity is maintained in the individual during the entire 14 day period.

Also provided herein are dosing regimens for administering a compound described herein (such as the compound of Formula 5) to an individual in need thereof. In some embodiments, the compound is administered to the individual once daily. In some embodiments, the compound is administered to the individual every other day. In some embodiments, the compound is administered to the individual every two days, every three days, every four days, every five days or every six days. In some embodiments, the compound is administered to the individual once per day for at least seven days. In some embodiments, the compound is administered to the individual once per day for at least 14 days. In some embodiments, the compound is administered to the individual once per day for a period of between one and four weeks.

In some embodiments, about 1 mg to about 10 mg of a compound described herein (such as the compound of Formula 5) is administered to the individual. The dosage amount of a compound as described herein is determined based on the free base of a compound, such as the free base of a compound of Formula 5. In some embodiments about 1 mg to about 5 mg of the compound is administered to the individual. In some embodiments about 1 mg to about 3 mg of the compound is administered to the individual. In some embodiments about 5 mg to about 10 mg of the compound is administered to the individual. In some embodiments, about 1 mg of the compound is administered to the individual. In some embodiments, about 2 mg of the compound is administered to the individual. In some embodiments, about 3 mg of the compound is administered to the individual. In some embodiments, about 4 mg of the compound is administered to the individual. In some embodiments, about 5 mg of the compound is administered to the individual. In some embodiments, about 6 mg of the compound is administered to the individual. In some embodiments, about 7 mg of the compound is administered to the individual. In some embodiments, about 8 mg of the compound is administered to the individual. In some embodiments, about 9 mg of the compound is administered to the individual. In some embodiments, about 10 mg of the compound is administered to the individual. In some embodiments, the compound is administered to the individual daily, such as once daily. In some embodiments, the compound is administered to the individual weekly. In one embodiment, the compound is a compound of Formula 5, or a pharmaceutically acceptable salt thereof, such as a tosylate salt.

In some embodiments, the compound is administered to the individual once per day for at least seven days in a daily amount of about 1 mg to about 10 mg or about 1 mg to about 5 mg or about 1 mg to about 3 mg or about any one of 1, 2, 3, 4, 5, 6, 7, 8, 9, or 10 mg. In some embodiments, the compound is administered to the individual once per day for at least 14 days in a daily amount of about 1 mg to about 10 mg or about 1 mg to about 5 mg or about 1 mg to about 3 mg or about any one of 1, 2, 3, 4, 5, 6, 7, 8, 9, or 10 mg. In some embodiments, the compound is administered to the individual once per day for a period of between one and four weeks in a daily amount of about 1 mg to about 10 mg or about 1 mg to about 5 mg or about 1 mg to about 3 mg or about any one of 1, 2, 3, 4, 5, 6, 7, 8, 9, or 10 mg. In any such embodiments, the daily administration in one embodiment is via inhalation. In any such embodiments, the daily administration in one embodiment is via oral dosing. In any such embodiments, the compound in one embodiment is the compound of Formula 5, or a tosylate salt thereof.

Also provided herein are methods of treating a respiratory disease comprising administering an effective amount of compound as provided herein in combination with a second agent. In some embodiments, the second agent is an anti-viral agent. In some embodiments, the second agent is an antibacterial agent. In some embodiments, the second agent is an influenza virus inhibitor, such as oseltamivir (OTV), sold under the brand name TAMIFLU. In some embodiments, the second agent is a nucleotide analog, such as remdesivir. In some embodiments, the second agent is an antiviral compound as described in De Clercq, E. & Li, G. Approved antiviral drugs over the past 50 years. Clin. Microbiol. Rev. 29, 695-747 (2016), which is incorporated herein by reference in its entirety and specifically with respect to the anti-viral compounds disclosed therein. In some embodiments, a compound provided herein is administered simultaneously with a second agent. In some embodiments, a compound provided herein, and the second agent are delivered sequentially.

### Articles of Manufacture and Kits

The present disclosure further provides articles of manufacture comprising a compound described herein, or a salt thereof, a composition described herein, or one or more unit dosages described herein in suitable packaging. In certain embodiments, the article of manufacture is for use in any of the methods described herein. Suitable packaging is known in the art and includes, for example, vials, vessels, ampules, bottles, jars, flexible packaging and the like. An article of manufacture may further be sterilized and/or sealed.

The present disclosure further provides kits for carrying out the methods of the present disclosure, which comprises one or more compounds described herein, or a pharmaceutically acceptable salt thereof, or a composition comprising a compound described herein or a pharmaceutically acceptable salt thereof. The kits may employ any of the compounds disclosed herein or a pharmaceutically acceptable salt thereof. In one variation, the kit employs a compound described herein or a pharmaceutically acceptable salt thereof. The kits may be used for any one or more of the uses described herein, and, accordingly, may contain instructions for the treatment of any disease or described herein, for example for the treatment of a respiratory disorder such as ARDS.

Kits generally comprise suitable packaging. The kits may comprise one or more containers comprising any compound described herein or a pharmaceutically acceptable salt thereof. Each component (if there is more than one component) can be packaged in separate containers or some components can be combined in one container where cross-reactivity and shelf life permit.

The kits may be in unit dosage forms, bulk packages (e.g., multi-dose packages) or sub-unit doses. For example, kits may be provided that contain sufficient dosages of a compound as disclosed herein, or a pharmaceutically acceptable salt thereof, and/or an additional pharmaceutically active compound useful for a disease detailed herein to provide effective treatment of an individual for an extended period, such as any of a week, 2 weeks, 3 weeks, 4 weeks, 6 weeks, 8 weeks, 3 months. 4 months. 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present disclosure. The instructions included with the kit generally include information as to the components and their administration to an individual.

### EXAMPLES

### Example 1

### Background

Semicarbazide-sensitive amine oxidase (SSAO) contributes to non-alcoholic steatohepatitis (NASH) by increasing oxidative stress through deamination of primary amines (e.g. methylamine, MMA) to aldehyde, ammonium, and H₂O₂ and by recruitment of inflammatory cells to the liver, exacerbating hepatic inflammation and injury. SSAO levels are elevated in NASH and correlate with fibrosis stage. The compound of Formula 5 is a selective, covalent SSAO inhibitor that decreases liver inflammation and fibrosis in a rat model of NASH. A single-ascending dose clinical trial of the compound of Formula 5 was performed.

The compounds described herein may be obtained by the methods described in WO 2018/028517, which is incorporated herein by reference in its entirety and specifically with respect to the methods of making the compounds detailed herein.

### Methods

Four groups of 8 healthy participants were randomized to receive the compound of Formula 5 capsule or matching placebo in a 3:1 ratio. Plasma levels of the compound of Formula 5 and PD biomarkers were determined at pre-dose and various time points post-dose. SSAO inhibition was determined by measuring relative reductions in plasma H₂O₂ generation after addition of an exogenous substrate (benzylamine). Endogenous methylamine (MMA) levels, predicted to increase upon SSAO inhibition, were measured in plasma. Safety was assessed for 7 (±3) days after dosing.

Plasma samples for the compound of Formula 5 concentration and SSAO activity determination were collected at 0.25, 0.5, 1, 2, 3, 4, 6, 8, 10, 12, 24, 48 (SSAO activity only), and 168 (SSAO activity only) hours after administration of a single dose of study medication (placebo or compound). Plasma PK parameters were determined by non-compartmental analysis. SSAO activity was assessed by measuring hydrogen peroxide (H₂O₂) generation levels in plasma samples from placebo and active compound of Formula 5 recipients. Percent change in total amine oxidase activity was determined relative to the corresponding pre-dose (baseline) samples.

SSAO-specific amine oxidase levels in plasma were determined using a kinetic-based assay essentially as described previously (Schilter et al). Endogenous monoamine oxidases A and B were inhibited by adding pargyline to plasma samples prior to measuring H₂O₂ generation levels in placebo and active recipients. Maximum inhibition was defined by pre-dose (baseline) samples additionally treated with a high dose of the compound of Formula 5 and percent changes in SSAO-specific activity were calculated relative to baseline samples.

### Results

32 healthy human participants (100% male, 63% Black, 19% Asian, 13% Caucasian) were enrolled and received a single oral dose of the compound of Formula 5 (1, 3, 6, and 10 mg, n=6 each) or placebo (n=2). The compound of Formula 5 plasma PK exposure increased in a greater than dose proportional manner between the 3 and 10 mg dose levels. The mean half-life of the compound of Formula 5 ranged from 1-3 hours. At 4 hours post-dose, near complete inhibition of plasma SSAO activity was seen in all dose cohorts and continued suppression was detected for up to 1 week after a single dose of the compound of Formula 5. Maximum plasma MMA levels increased with the compound of Formula 5. No clinically relevant adverse events or laboratory abnormalities were reported.

As shown in Table 1, doses 1, 3, 6, and 10 mg of the compound of Formula 5 were all well tolerated.

TABLE 1
Treatment Associated Adverse Events

| | 1 | 2 | | 4 | |
|---|---|---|---|---|---|
| | 1 mg of a | 3 mg of a | 3 | 10 mg of a | |
| | toslyate salt | toslyate salt | 6 mg of a | toslyate salt | |
| | of Formula 5 | of Formula 5 | toslyate salt | of Formula 5 | |
| | or placebo | or placebo | of Formula 5 | or placebo | All |
| | (n = 8) | (n = 8) | (n = 8) | (n = 8) | (n = 32) |
| Subject incidence | 0 | 0 | 2 (25) | 3 (37.5) | 5 (15.6) |
| of any TEAE | | | | | |
| Subject incidence | 0 | 0 | 0 | 1 (12.5) | 1 (3.1) |
| of TEAEs | | | | | |
| considered possibly | | | | | |
| treatment-related | | | | | |
| TEAE diagnosis | | | | | |
| and frequency | | | | | |
| constipation | 0 | 0 | 0 | 1 (12.5) | 1 (3.1) |
| contact dermatitis | 0 | 0 | 2 (25) | 0 | 2 (6.3) |
| dysgeusia | 0 | 0 | 0 | 1 (12.5) | 1 (3.1) |
| headache | 0 | 0 | 0 | 1 (12.5) | 1 (3.1) |
| oral herpes | 0 | 0 | 0 | 1 (12.5) | 1 (3.1) |
| sore throat | 0 | 0 | 1 (12.5) | 0 | 1 (3.1) |
| upper respiratory | 0 | 0 | 0 | 1 (12.5) | 1 (3.1) |
| tract infection | | | | | |

Single doses of the tosylate salt of compound of Formula 5 were rapidly cleared from plasma and exhibited greater than dose proportional plasma PK between 3 and 10 mg.

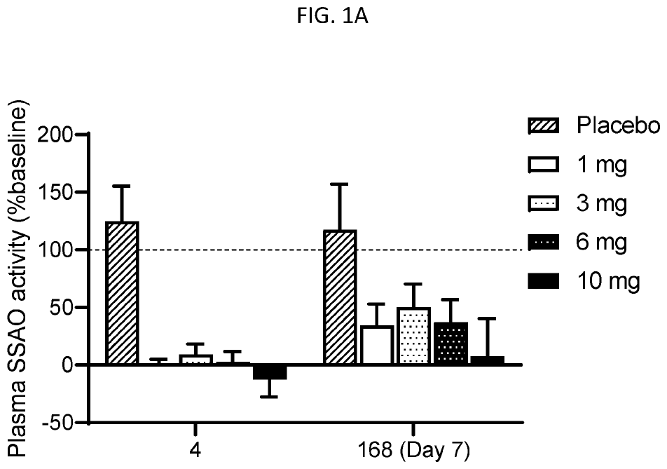

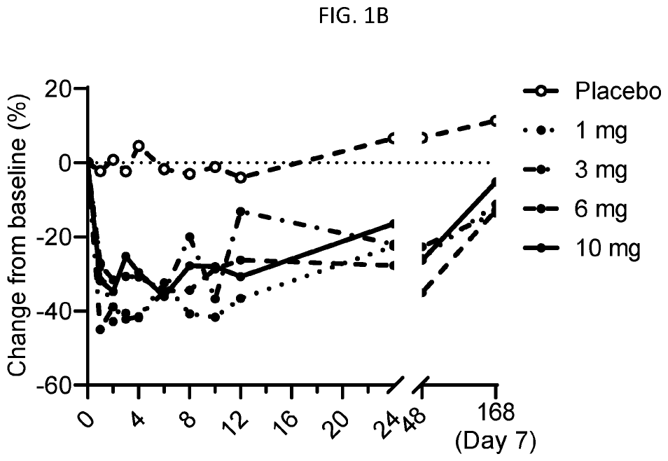

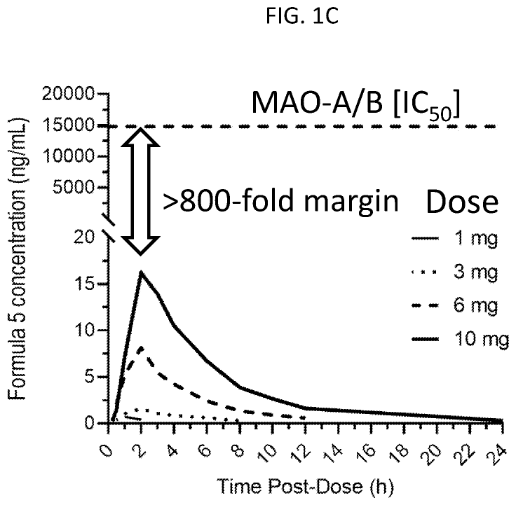

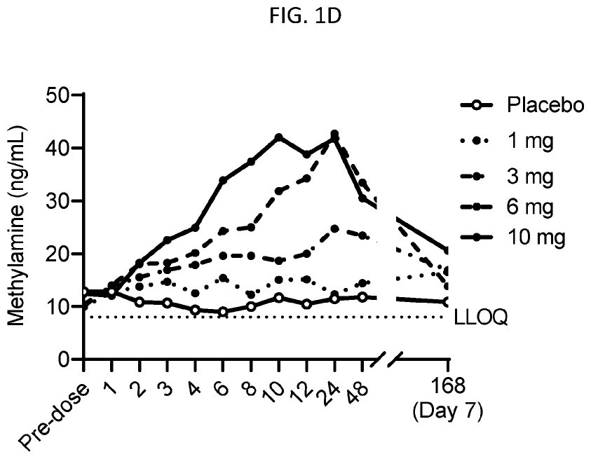

Single doses of the compound of Formula 5 rapidly and potently decreased plasma amine oxidase activity in all subjects as shown in FIG. 1A and FIG. 1B. Near complete inhibition of SSAO-specific activity was observed at 4 hours post dose (FIG. 1A and FIG. 1B). Inhibition of plasma SSAO amine oxidase activity and dose-dependent increases in plasma MMA were sustained up to 1 week after single doses of the compound of Formula 5, suggesting potent, covalent target engagement and supporting once daily dosing despite a short plasma half-life (FIG. 1A and FIG. 1B).

The concentrations (Cₘₐₓ) for the compound of Formula 5 were more than 800 times lower than the IC₅₀ concentrations for MAO-A and MAO-B at all dose levels.

TABLE 2
Biochemical activity (IC₅₀ μM)

| SSAO inhibitor | SSAO | MAO-A | MAO-B |
|---|---|---|---|
| Compound of | 0.0065 | >50 | >50 |
| Formula 5 | | | |
| BI 1467335 | 0.005 | >100 | 2.7 |
| (PXS-4728A) | | | |

Dose-dependent increases in methylamine were observed, indicating potent plasma SSAO target engagement across the dose range (FIG. 1D).

### Conclusions

The compound of Formula 5 was safe and well tolerated in healthy subjects administered a single oral dose ranging from 1 mg to 10 mg. The compound of Formula 5 inhibited SSAO activity for up to seven days after a single dose. This suggests that the compound of Formula 5 may be effective for treating diseases or disorders such as respiratory diseases by selectively inhibiting SSAO. It may also exhibit SSAO activity for seven days after only a single dose, suggesting that daily administration for one week may exert a therapeutic effect for a two-week period.

### Example 2

A Phase 2a proof-of-concept study for the compound of Formula 5 in COVID-19 patients with severe pulmonary dysfunction is conducted. Concomitant pre-clinical and clinical pharmacodynamic and biomarker studies are conducted. Experiments are conducted to test the effect of a compound of Formula 5 in acute and chronic lung injury. ARDS, and virus-induced lung injury models.

### Example 3

Experiments are conducted to test whether the compound of Formula 5 can suppress infiltration of inflammatory cells in the lungs as in Schliter et al. Respiratory Research 16:42 (2015). Briefly, BALBc mice are anesthetized and given an immunogenic composition, such as lipopolysaccharide, Klebsiella pneumoniae, dust mite (Dermatophagoides pternyssinus), rCXCL1. Cecal ligation and puncture is also performed to induce sepsis.

Mice are administered the compound of Formula 5. At time points, measurements are taken to assess the PK/PD of the compound of Formula 5 and the effect of the compound on inflammation in the lung.

Levels of MAO-A, MAO-B, DAO, LOXL2, and LOX are measured using florigenic assays. Total cells and neutrophils in the lungs are measured. Cell adhesion and rolling are measured. Survival is also measured.

### Example 4

Senescent mice infected with coronavirus are used as a model for virus-induced ARDS as in Chen et al., J. Virology 84(3)1298-1301 (2010). Briefly, mouse-adapted SARS-CoV is propagated in Vero cells, with a titer of 106.5 50% tissue culture infective doses. Vero cells are maintained in OptiPro SFM (Invitrogen, CA). Female BALB/c mice. 12 to 14 mo, are administered 105 TCID50 of mouse-adapted SARS-CoV intranasally (i.n.).

Mice are administered the compound of Formula 5. Lungs are harvested at serial time points. Histopathology, morbidity, and mortality are assessed.

### Example 5

A mouse-adapted SARS coronavirus challenge model is used to assess the efficacy of a compound of Formula 5.

The methods in Sheahan et al. Sci. Transl. Med. 9 (Jun. 28, 2017) are used. Briefly, mice are infected with 10e4 PFU/50 mL (prophylactic studies) or 10e3 PFU/50 mL (therapeutic studies) mouse-adapted SARS-CoV MA15. Roberts et al. Plos Pathogens (2007) January; 3(1). Animals are weighed daily to monitor virus associated weight loss.

Mice are administered the compound of Formula 5.

At time points, mice are sacrifice and the level of virus is detected using a viral plaque assay or viral RNA quantitative methods.

### Example 6

A mouse lethal challenge model of influenza A virus infection was used to assess the efficacy of a compound of Formula 5.

Instrumentation: The main instruments used in this study included electronic balance, biosafety cabinet-II and centrifuge (Thermo & Fisher), CO2 incubator (Thermo), Cell count (Invitrogen), tissue lyser II (Qiagen), Nanodrop 1000 (Thermo Scientific), Microplate reader SpectraMax (Molecular Devices), Luminex (BIO-RAD), blood analyser (Siemens).

Reagents: The key reagents used in this study included MDCK cell, DME medium, Ultra MDCK Serum-free Medium, paraformaldehyde, crystal violet, mouse cytokine detection kit (Th1/Th2 Cytokine 11-Plex Mouse ProcartaPlex™ Panel) and Hydroxyproline quantitation kit. Oseltamivir phosphate was used as the positive control. PBS was used as the vehicle and solvent of a compound of Formula 5 and oseltamivir phosphate.

Formulation: The oseltamivir was dissolved in PBS at the concentration of 0.5 mg/mL and 1.0 mg/mL. The compound of Formula 5 was dissolved in the solvent indicated above at the concentrations of 2.0 mg/mL and 4.0 mg/mL. Clear solutions were obtained by ultra-sonication and stirring. Daily prepared and stored at 4 degree C. before use.

Virus: Influenza A virus, WSN/33 strain of H1N1, 3.0×106 p.f.u./mL

### In-Life Methods

144 female 6-8 week old pathogen free BALB/c mice were divided into 16 groups representing healthy control (1 group), vehicle (1 group), oseltamivir monotherapy (positive control, 2 groups), a compound of Formula 5 monotherapy (2 groups), and oseltamivir and a compound of Formula 5 combination treatment (2 groups), with 8 or 10 mice per group. The 10-mice cohort was used for sample collection on day 6, and the 8-mice cohort was used for body weight and mortality monitoring. The mice were treated with vehicle or test compounds following the regimen of PO from day 0 or day 1 to day 5 or day 6, with first dose given at 2 hr prior to inoculation (prophylactic group) or 36 hr after inoculation (treatment group) of a lethal challenge with influenza A virus WSN/33 strain at inoculation dose of 15,000 p.f.u./mouse/50 μL delivered via intranasal route under general anesthesia. All mice were monitored daily for body weight loss (BWL), health status, and mortality.

Mice in the sample collection arms were sacrificed on day 6 for lung viral titration and histology evaluation; bronchoalveolar lavage fluid (BALF) samples were harvested for cytokine detection and immune cell counting; blood was harvested via submandibular vein puncture for plasma collection.

Mice in the monitoring arm were monitored through the end of study. Surviving mice were sacrificed on day 14 and lung samples were harvested for hydroxyproline quantitation and lung fibrosis analysis as a measure of in viva efficacy. Kidney and plasma were also collected. Plasma samples were obtained from K₂EDTA anticoagulated blood samples by centrifuging at 7,000×g, 4° C. for 10 minutes. Plasma samples were stored at −80° C. until transferred for further assays.

TABLE 3
Study design and schedule.

| | | | | | Dose | |
|---|---|---|---|---|---|---|
| | | | | Dosage | Volume | Dosing Route |
| | Group | Animals | Compound ID | (mg/kg) | (mL/kg) | and Regimen |
| | 1 | 8 | NA | NA | NA | NA |
| | 2 | 10 | | | | NA |
| | 3 | 8 | Vehicle | 0 | 10 | PO, QD, day 0-day 6, first |
| | | | | | | dose given at −2 hr P.I. |
| | 4 | 10 | | | | PO, QD, day 0-day 5, first |
| | | | | | | dose given at −2 hr P.I. |
| PROPHYLACTIC | 5 | 8 | Compound of | 20 | 10 | PO, QD, day 0-day 6, first |
| | | | Formula 5 | | | dose given at −2 hr P.I. |
| | 6 | 10 | | | | PO, QD, day 0-day 5, first |
| | | | | | | dose given at −2 hr P.I. |
| | 7 | 8 | Oseltamivir | 5 | 10 | PO, BID, day 0-day 6, first |
| | | | | | | dose given at 2 hr P.I. |
| | 8 | 10 | | | | PO, BID, day 0-day 5, first |
| | | | | | | dose given at −2 hr P.I. |
| | 9 | 8 | Compound of | 20 + 5 | 5 + 5 | PO, QD/BID, day 0-day 6, |
| | | | Formula 5 + | | | first dose given at −2 hr P.I. |
| | 10 | 10 | Oseltamivir | | | PO, QD/BID, day 0-day 5, |
| | | | | | | first dose given at 2 hr P.I. |
| TREATMENT | 11 | 8 | Compound of | 20 | 10 | PO, QD, day 1-day 6, first |
| | | | Formula 5 | | | dose given at +36 hr P.I. |
| | 12 | 10 | | | | PO, QD, day 1-day 5, first |
| | | | | | | dose given at +36 hr P.I. |
| | 13 | 8 | Oseltamivir | 5 | 10 | PO, BID, day 1-day 6, first |
| | | | | | | dose given at +36 hr P.I. |
| | 14 | 10 | | | | PO, BID, day 1-day 5, first |
| | | | | | | dose given at +36 hr P.I. |
| | 15 | 8 | Compound of | 20 + 5 | 5 + 5 | PO, QD/BID, day 1-day 6, |
| | | | Formula 5 + | | | first dose given at +36 hr P.I. |
| | 16 | 10 | Oseltamivir | | | PO, QD/BID, day 1-day 5, |
| | | | | | | first dose given at +36 hr P.I. |

PO, oral administration;
QD, once per day;
BID, twice per day;
P.I., post-infection

### Post-Life Methods

Viral titration: The lung viral titer was determined by plaque assay with the following procedures.

a). Cell seeding: MDCK cells were seeded in the 6-well plate with 1.67×10⁵/mL, 3 mL/well. The plates were incubated in the CO₂ incubator overnight to get the monolayer cells.
b). Sample processing: lung samples were homogenized with the tissue lyser. Supernatant of the lung homogenate was obtained by centrifugation, and then 10-fold serially diluted with medium.
c). Inoculation: 0.1 mL supernatant and the serial dilutions were pipetted into the seeded 6-well plate for 4 hours' absorbing.
d). Incubation: The liquid medium was removed and replaced with the low-melting agarose medium. The plates were put in the CO2 incubator for 3 days after the agarose was solidified.
e). Fixation and staining: The cells were fixed with 4% paraformaldehyde and stained with 0.5% crystal violet for counting.
f). Plaques counting: The plaques in the photos were visually counted and the viral titer was calculated, which was expressed as Log₁₀ (plaques/g lung tissue)=Log₁₀ (plaques/well*1000*dilution factors).

Cytokines detection: The GM-CSF. IFN gamma, IL-1 beta, IL-12p70, IL13, IL-18, IL-2, IL-4, IL-5, IL-6 and TNF alpha levels in BALF were detected by Luminex with the kit above mentioned by following the protocol and the operation manual provided by the manufacturer.

Cell counting in BALFs: White blood cells, neutrophil, lymphocyte, and monocyte in the BALF samples were tested by the blood analyzer by following the operation manual.

Lung histology detection: Lung histology and fibrosis were analyzed by HE/Masson staining and scoring.

a). Dehydration of tissues: all tissue samples fixed in 10% Neutral Buffer Formalin (NBF) for 24˜48 hrs were trimmed for about 3˜5 mm thickness, placed in the embedding boxes, and dehydrated in the tissue processor.
b). Paraffin embedding and tissue section: all dehydrated tissues were embedded with paraffin using a tissue embedder and the finished paraffin blocks were stored at room temperature (RT) until section.
c). H&E staining procedure: before staining, all tissue sections were warmed at 60° C. for at least 45 minutes. Thereafter the samples were set in the auto-staining machine and proceed the pre-set procedures.
d). Histopathology evaluation: the slices were examined by a pathologist and scored for lung injury.

Hydroxyproline quantitation: Hydroxyproline was determined with the commercial kit by following the protocol and the operation manual provided by the manufacturer.

### Data Analysis

The data was statistically analyzed to see the differences of results between vehicle and the test articles, p>0.05, no significant difference, p<0.05, significant difference, p<0.01, extremely significant difference. The BWL was analyzed by Two Way ANOVA. The survival analysis was conducted by Log-rank. The lung viral titer, BALF cytokines level, hydroxyproline level and lung pathology analysis were conducted by One Way ANOVA.

The in vivo efficacy of the test articles were determined by the BLW, survival proportions as well as the lung viral titer. Immune cell counting and the cytokines level were used to evaluate the effect on anti-inflammation. HE staining was used for lung pathology evaluation. Masson staining and hydroxyproline detection were used for lung fibrosis evaluation.

### Results

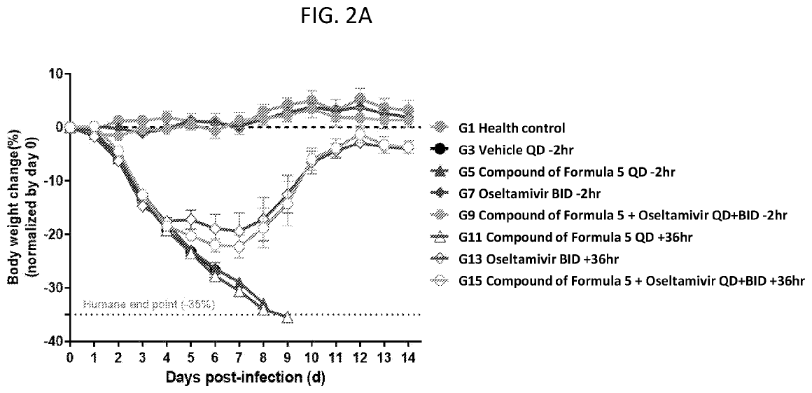

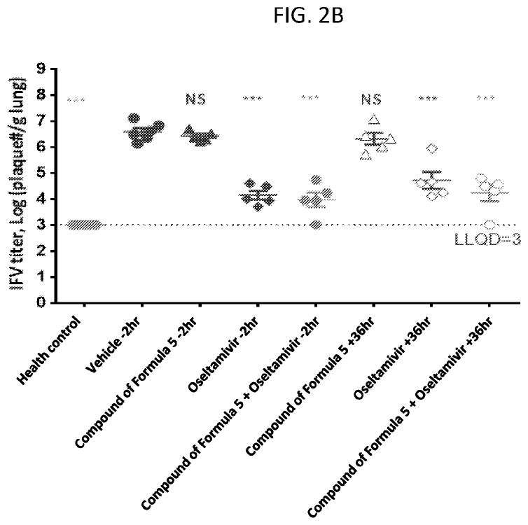

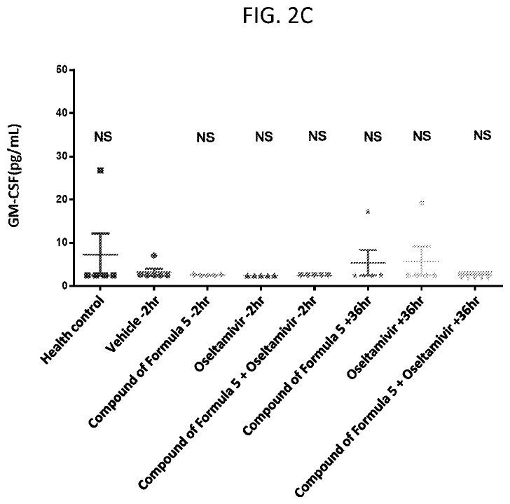

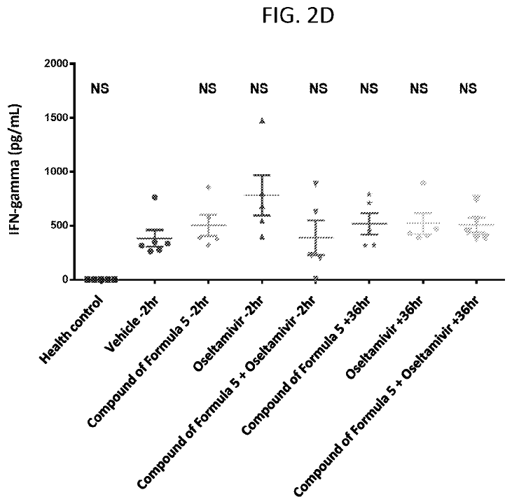

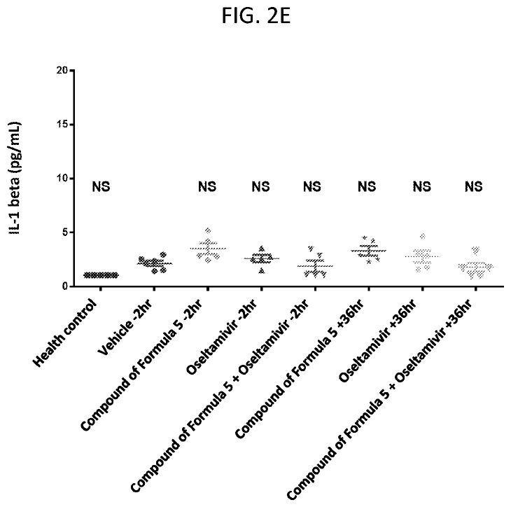

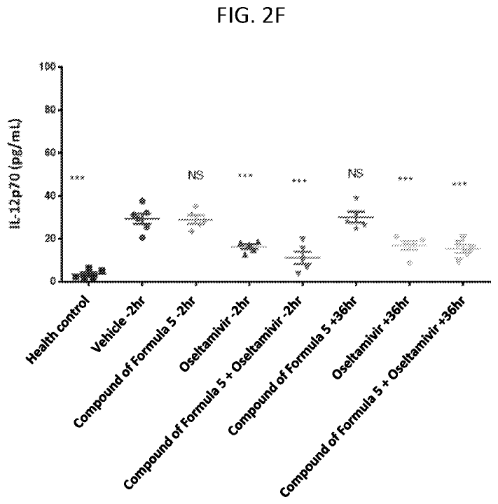

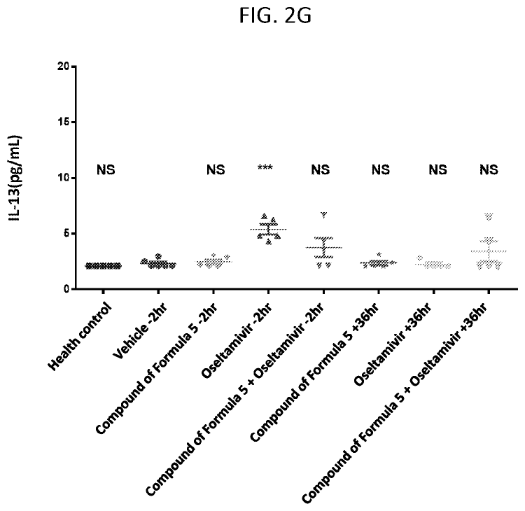

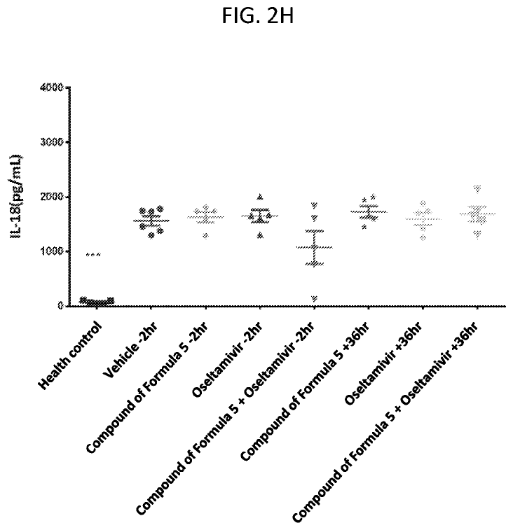

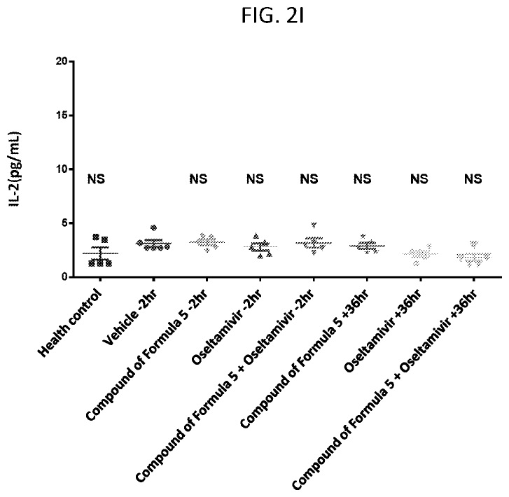

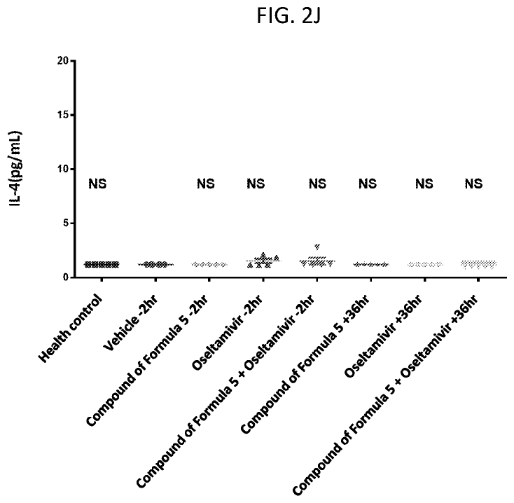

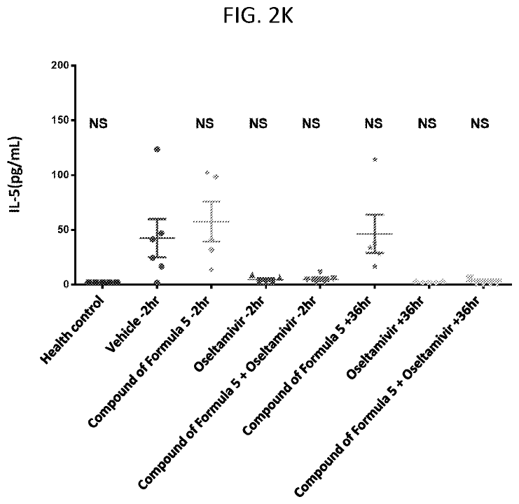

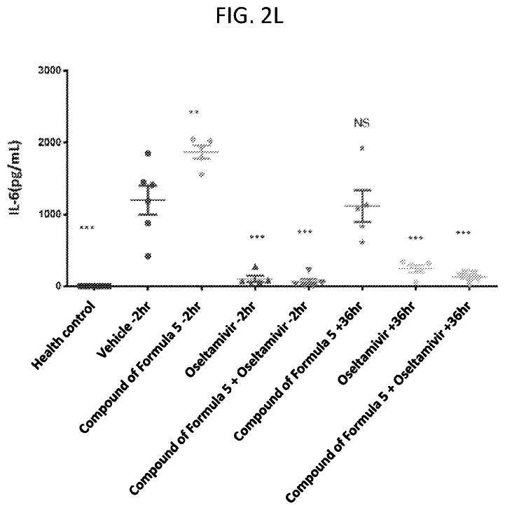

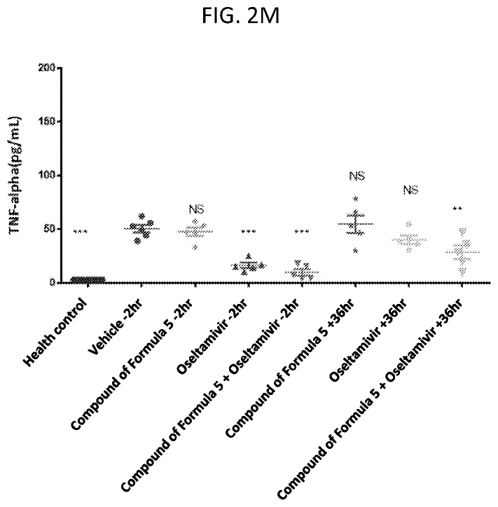

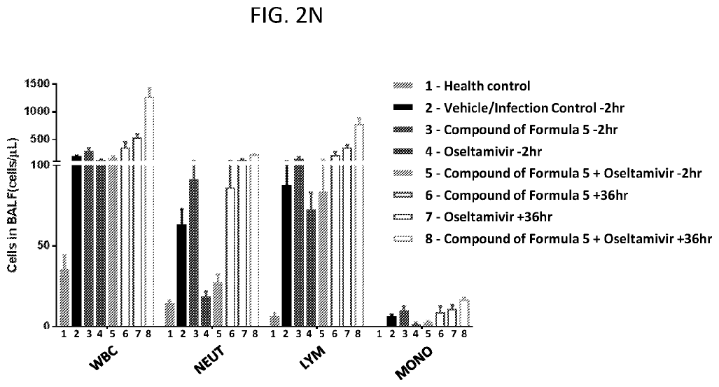

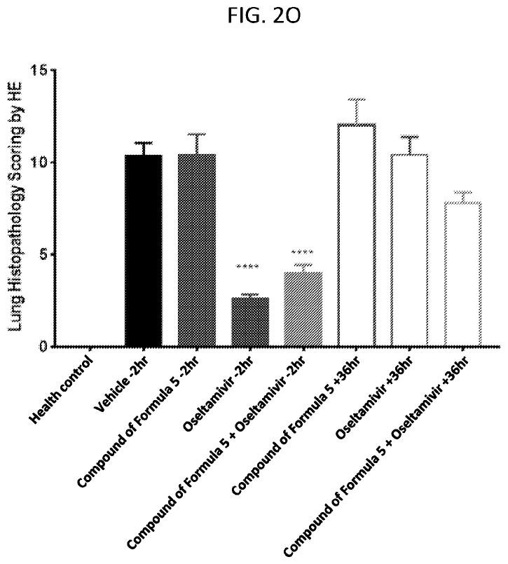

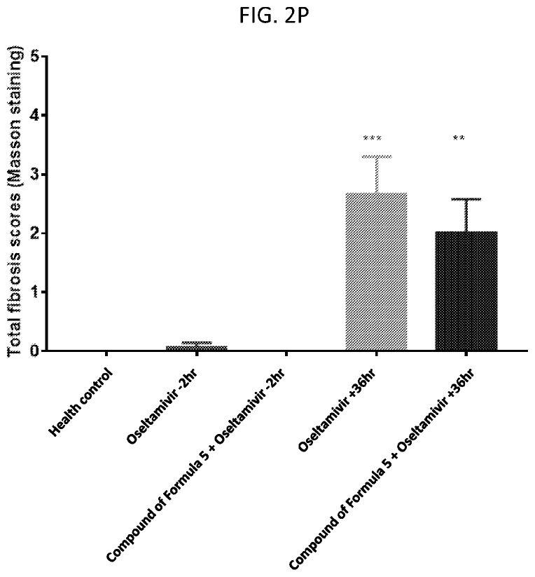

During the entire period of the in-life study, the mice were monitored daily for body weight. The body weight loss (BWL), which was normalized by the day 0 body weight (FIG. 2A).

TABLE 4
Surviving Status

| | | | | Median | | |
|---|---|---|---|---|---|---|
| | | Treatment | Survival | survival | Prolonged | |
| | Treatment | Dosage | proportion | time | survival | Log-rank |
| Group | Compound | (mg/kg) | (%) | (day) | time* (%) | P value* |
| 1 | NA | 0 | 100 | undefined | undefined | <0.0001 |
| 3 | Vehicle | 0 | 0 | 5 | NA | NA |
| 5 | Compound of | 20 | 0 | 6 | 20% | 0.2280 |
| | Formula 5 | | | | | |
| 7 | Oseltamivir | 5 | 100 | undefined | undefined | <0.0001 |
| 9 | Compound of | 20 + 5 | 100 | undefined | undefined | <0.0001 |
| | Formula 5 + | | | | | |
| | Oseltamivir | | | | | |
| 11 | Compound of | 20 | 0 | 6 | 20% | 0.1041 |
| | Formula 5 | | | | | |
| 13 | Oseltamivir | 5 | 100 | undefined | undefined | <0.0001 |
| 15 | Compound of | 20 + 5 | 87.5 | undefined | undefined | <0.0001 |
| | Formula 5 + | | | | | |
| | Oseltamivir | | | | | |

*compared with vehicle group

Lung Viral Titer Detection: All the mice were treated as indicated and sacrificed on day 6 for lung viral titration. The titration was performed by plaque assay. The data is shown as Mean±SEM and analyzed by a plaque assay (FIG. 2B). Unless otherwise specified, N=5 (N=6 for vehicle group). Data were analyzed by One Way ANOVA, corrected by Dunnett's multiple comparisons test. NS, not significant (p>0.05); ***p<0.001 compared with vehicle group.

BALF cytokine profiling: BALF cytokine level (granulocyte-macrophage colony-stimulating factor, GM-CSF; IFN-gamma, IL-1 beta, IL-12p70, IL13, IL-18, IL-2, IL-4, IL-5, IL-6 and TNF alpha) was detected to evaluate the immunomodulatory effects of a compound of Formula 5 treatment in the IAV infection mouse model (FIG. 2C-2M).

Cell counting in BALF (FIG. 2N; WBC=white blood cells; NEUT=neutrophil; LYM=lymphocyte; MONO=monocyte).

Pathology Evaluation: Lung histopathology scoring was scored on day 6 by HE staining for each category identified in Table 5. The sum of scores across categories was plotted in FIG. 2O.

TABLE 5
Lung histology scoring

| Tissue/Organ | Histopathology changes |
|---|---|
| Lung | Conducting tract (Airway) |
| | Necrosis/Exfoliation ^{a}, epitheliums of the |
| | bronchioles/terminal bronchioles |
| Respiratory zone | Edema, alveolus ^{b} |
| | Hyaline membrane formation, alveolar duct/sac |
| | Dilation, alveolar duct/sac |
| | Inflammatory infiltration (macrophages, |
| | lymphocytes, neutrophils), alveolus |
| | Hemorrhage, alveolus |
| Interstitium | Inflammatory infiltration (macrophages, |
| | lymphocytes, neutrophils), interstitial |
| | (peri-bronchiloes predominant, alveolar |
| | septa, perivascular ^{d}) |
| | Congestion, alveolar septa |
| | Edema, perivascular |
| | Hemorrhage, perivascular |

Scoring: 1, minimal; 2, mild; 3, moderate; 4, marked; 5, severe; N, No visible lesion.
^{a} this change was accompanied by cellular debris in the lumen of affected bronchioles;
^{b} mainly observed in the alveolar space beneath the pulmonary pleura;
^{d} leukocytes adhesion was observed in the intima of the vessels;
^{e} numerous scattered cellular debris were noted in the interstitium.

Fibrosis evaluation. Masson staining was conducted. Scoring: 0=Normal lung; 1 Minimal collagen fibers in the thickening of alveolar or bronchiolar walls; 2/3 mild or moderate thickening of walls without obvious damage to lung architecture; 4/5 Increased fibrosis with definite damage to lung structure and formation of fibrous bands or small fibrous masses; 6/7 Severe distortion of structure and large fibrous areas; 8 Total fibrous obliteration of the field. ** P<0.01; *** p<0.001 compared to healthy control. Error bars represent SEM (FIG. 2P).

Hydroxyproline detection: Hydroxyproline levels were measured in BALF samples harvested on day 14 and were below the lower limit of quantification (0.2 ug/uL).

100% of the mice in the combination prophylactic group survived to the end of the study, and 87.5% for the combo therapeutic group (Table 4). Combination therapeutic and prophylactic treatment showed significantly reduced lung viral titers (FIG. 2B). Combination therapeutic and prophylactic treatment lowered the levels of IL-12p70 (FIG. 2F), IL-6 (FIG. 2L), and TNF alpha (FIG. 2M) in BALF samples. Combination prophylactic treatment showed lower NEUT number counting (FIG. 2N). Combination prophylactic treatment showed significant reductions in pathology injury by HE staining, and moderate improvements were found in combination therapeutic treatment group (FIG. 2O). Combination prophylactic treatment group showed moderate decreases in pulmonary fibrosis as measured by histological evaluation of lung tissue by Masson staining (FIG. 2P).

Taken together, these data suggest that oseltamivir therapy in combination with a compound of Formula 5 was effective at reducing morbidity and mortality in a mouse lethal challenge model of influenza A virus infection. A compound of Formula 5 treatment, either as a monotherapy or in combination with oseltamivir, did not appear to exacerbate viral replication or pathology in this model.

All publications, including patents, patent applications, and scientific articles, mentioned in this specification are herein incorporated by reference in their entirety for all purposes to the same extent as if each individual publication, including patent, patent application, or scientific article, were specifically and individually indicated to be incorporated by reference.

Although the foregoing invention has been described in some detail by way of illustration and example for purposes of clarity of understanding, it is apparent to those skilled in the art that certain minor changes and modifications will be practiced in light of the above teaching. Therefore, the description and examples should not be construed as limiting the scope of the invention.

What is claimed is:

1. A method of treating a respiratory disease comprising administering an effective amount of a compound of Formula 5, or a pharmaceutically acceptable salt thereof to an individual in need thereof.

2. The method of claim 1, wherein the respiratory disease is acute respiratory distress syndrome (ARDS).

3. The method of claim 1 or 2, wherein inflammation in the lungs of the individual is reduced.

4. The method of any one of claims 1-3, wherein the level of neutrophils in the individual is reduced.

5. The method of any one of claims 2-4, wherein the individual has acute respiratory distress syndrome caused by a pathogen.

6. The method of claim 5, wherein the pathogen is a virus or bacteria.

7. The method of claim 5, wherein the pathogen is selected from the group consisting of SARS-CoV-2, SARS-CoV, MERS-CoV, Influenza virus, Metapneumovirus, Varicella zoster virus, Streptococcus pneumoniae, Haemophilus influenzae, Mycobacterium tuberculosis, COVID-19 and Malaria.

8. The method of any one of claims 1-7, wherein the compound is administered to the individual once daily.

9. The method of any one of claims 1-8, wherein the compound is administered at a dose of about 1 mg to about 10 mg.

10. The method of any one of claims 1-9, wherein MAO-A and MAO-B are not inhibited.

11. The method of any one of claims 1-10, wherein the compound is delivered intranasally or via inhalation.
